(19) United States
(12) Patent Application Publication
(10) Pub. No.: US 2004/0023264 A1
(43) Pub. Date: Feb. 5, 2004

(54) Method for determining the peptide hormone activities or the steroid hormone activities of a material or substance mixture

(76) Inventors: Axel Allera, Lohmar (DE); Ludwig Wildt, Herzogenaurach (DE)

Correspondence Address: FRISHAUF, HOLTZ, GOODMAN & CHICK, PC, 767 THIRD AVENUE, 25TH FLOOR, NEW YORK, NY, 10017-2023, US

(21) Appl. No.: 10/380,963

(22) PCT Filed: Sep. 20, 2001
(86) PCT No.: PCT/DE01/03628

(30) Foreign Application Priority Data
Sep. 20, 2000 (DE) 100 46 647.8

(51) Int. Cl. C12Q 1/68; C12N 5/06; C07K 14/72; C12N 15/85

(52) U.S. Cl. 435/6; 435/455; 435/325; 530/350

(57) ABSTRACT

The invention relates to a method for determining the peptide hormone activities or the steroid hormone activities of a material or substance mixture involving the following steps: (a) presenting at least one starting cell with or without endogenous peptide hormone receptors or steroid hormone receptors; (b) transfecting the cell with a specific recombinant reporter gene construct, which has the ability to express a product that is induced and can be measured by the hormone activity of the material; (c) introducing a material to be examined into the transfected cell; (d) producing an easily determinable signal-generating reporter gene translation product through the transfected cell by using the inductive effect of the material on the hormone-reactive promoter situated before the reported gene; (e) measuring the reporter gene translation product of the transfected cell, and; (f) determining the hormone activity of the material from the measured result.

This invention concerns a method of qualification and quantification of agonistic and antagonistic hormone activities of a material, a bioassay, and use of it.

Hormones are bioactive signalling molecules that are synthesised in specialised, so-called endocrine cells of any organism and, as a rule, are passed into the blood stream. This results in specific biological effects through the high affinity and highly specific reversible binding of these hormones to receptors of target cells which then transfer the hormone signal into the cell. Apart from peptide hormones (e.g. insulin, thyroid hormones, adrenaline), the most important hormones of almost all organisms are steroid hormones, which are mainly comprised of glucocorticoids, mineralocorticoids, androgens (incl. androgen anabolic agents or “doping” agents), estrogens and gestagens. They control growth, physical development and are essential for the course and maintenance of vital physiological and biological functions, e.g. reproduction.

The daily clinical routine of determination of concentration of the various peptide and steroid hormones—in natural and synthetic form—in serum and other bodily fluids of patients is an important factor in diagnosis. Hormones are used in the treatment of many disorders and diseases as well as in the regulation of physiological processes (e.g. the oral contraceptive pill). Widespread improper use is made of certain steroids, e.g. the various synthetic androgen derivatives that act as anabolic agents. Many top athletes take these as “doping” agents and detection of these agents in serum is used in doping control.

Also, considerable amounts of steroids are used in animal feed—quick and unproblematic determination of these, often illegal agents is of high importance, as they can enter the food chain for human consumption. In terms of the biological activity of peptide and steroid hormones—as agonist as well as antagonist—it must be noted that not only peptide and steroid hormones act biologically, i.e.,—chemically defined—as amino acid ligo- and polymer-derivatives the active materials correspond only in part or not at all with these chemical substance groups, but which nevertheless obviously bind to the respective hormone receptors.

The biological effects of such a material are also of high importance as it may result in, for example, basic problems within the biological signalling chain. These effects has been recognized in various softening agents, pesticides etc. Numerous substances of the natural and technological environment are of specific significance as these are estrogen active without chemically belonging to the steroid group. Individual components in material mixtures may even act synergistically in combination. It is therefore most desirable to enable determination of hormonal activity of such a material prior to or during the use of such a material.

Below, the invention will be explained in detail using as an example the determination of the biological effects of steroid hormones, especially glucocorticoid, androgen and estrogen active steroids. However, it must be noted that employment of this invention is not restricted to steroid hormones. In fact, it may be used universally for determination of typical steroid and peptide hormone effects of materials.

It is known that the biological activity of many materials, hormones in particular, does not necessarily equal its absolute concentration. Often, subsets of these substances have become inactive or more active as they may have formed complexes with proteins or similar or taken on a different configuration. The effect of thyroid hormones, for example, is known to vary depending on the extend of their binding to proteins. Other parameters—e.g. pH value or metal ions—may also strongly influence the effect of materials in humans or animals. Therefore, a distinction must be made between “free” and “bound” hormones and their components which then determine activity.

A decisive factor for determination of a so-called hormonal status and possibly its dynamic as well as the resulting diagnostic and therapeutic consequences is therefore not the quantity reconcentration of the respective peptide or steroid hormone but its activity, i.e. type and extent of biological effect. This is influenced, for example, by disassociation constants of ligand receptor complexes, but also many other factors, such as presence of transport proteins etc.

To start with, for better understanding of the invention and as an illustration, the effects of steroid hormones are explained. Steroid hormones transmit their effects mainly via receptors which are specific for the respective steroid hormone groups. According to the present state of research, these receptors are located within he target cells (the receptors for peptide hormones are located on or in the cell membrane, i.e. on the cell's surface). In doing so, they form a high affinity binding receptor steroid complex. This complex formation is an essential link of the hormone dependent signalling chain resulting in the hormone specific effect (see below).

Examples of steroid hormone receptors are—and this list is not restrictive, but for explanation purposes only: the glucocorticoid receptor (GR) which binds glucocorticoid cortisol; the androgen receptor (AR) which binds the male sexual hormone testosterone as well as various synthetic steroid hormone analogues such as anabolic agents; the estrogen receptors ER□ and ER□ which bind the female sexual hormone estradiol and synthetic estrogens (e.g. components of the contraceptive pill) as well as other materials with estrogen activity; the mineralocorticoid receptor (MR) or the progesterone receptor (PR) which recognizes aldoesterone and natural as well as synthetic gestagens, respectively. Following binding of the steroid to it, the receptor moves to the nucleus and binds onto specific DNA sequences of the nucleus, the so-called “hormone responsive elements” (HRE). Next, the HRE, which are components of a so-called promotor, regulate locally adjoining genes (so-called “downstream” genes). These downstream genes encode for specific proteins (e.g. enzymes). For example, the steroid hormone cortisol induces the essential enzyme tyrosinaminotransferase in liver cells; estradiol induces the PR or transcortin (an essential transport protein for glucocorticoids in blood). Surprisingly, at present, only determination of concentration of steroid hormones or anabolic agents is carried out—as part of, inter alia, diagnosis and doping control. Determination of steroid hormone activity has to date not been the norm and respective determination methods are now known, despite the fact that these would be more appropriate in biological and medical respects.

Burdhe CB et al.: “Determination of oestrogen concentration in bovin plasma by a recombinant oestrogen receptor-reporter gene yeast bioassay”, Analyst, 123 (1998), 2585-2588, suggest determination of estrogen concentration in bovine a rum via a yeast cell line which was stably transfected with an estrogen receptor galactosidase receptor construct (“Recombinant Cell Yeast Bioassay—RCBA”). Here, again, as is the rule in general clinical diagnosis, only the concentration of a single steroid hormone, in this case 17□-estradiol, is determined.

The purpose of the invention is to outline methods for the determination of hormone activity of a material.

This task is solved via a process according to the requirements for patent claim No. 1. Advantageous development ensues from further dependent claims. This invention also concerns a determination assay for hormone activity of materials according to patent claim No. 9.

It is implemented as follows:

1. Selection and presentation of a suitable cell—either

a) with endogenous hormone receptor, transiently or stably mono- or co-transfected with a recombinant hormone sensitive receptor gene construct (reporter expression plasmid) or additionally with a recombinant specific hormone receptor expression plasmid, or

b) without endogenous hormone receptor, transiently or stably cotransfected with a recombinant hormone sensitive reporter gene construct and a recombinant specific hormone receptor expression plasmid, which is then able, as transfected cell, to express—In case of a) or b)—a signalling, easily measurable product.

2. Input of a material or material mixture with known or assumed hormone activity into a transfected cell, generally via addition to the cell culture medium.

3. Induction of the cell's signalling reporter gene translation product caused by the material via the hormone receptor.

4. Measuring of the above-mentioned product, if necessary, following suitable preparation of the transfected cell according to generally known method.

5. Analysis: the quantity of the translation product formed (=induced) is directly proportional to the intensity of the respective hormone activity of the material examined.

For this application, transient transfection means input of foreign DNA into the cell's cytoplasm for the duration of one cell cycle. Stable transaction means permanent input of foreign DNA into its genome. Generally, genetically altered (recombinant) plasmids (“cloning vectors”) are used as vehicles, which contain the DNA sequence(s) for he desired reporter protein or the specific hormone receptor. These genes are preceded (“upstream”) by a specific promotor. This is either a constitutive promotor (e.g. with SV40 or Tk (thymidinkinase) in the receptor expression plasmid) causing permanent expression, or an inducible promotor (e.g. with HRE in the reporter expression plasmid) causing stimulated expression (e.g. by a hormone) of the adjoining reporter gene.

High transfection efficiency is obviously an important condition for reliable employment of the invention. At present, the preferred transfection method is the so-called highly effective “Non-Adenovirus-Polylysine” technique, published, or example, by Sommer B et al,: “Efficient gene transfer into normal human skeletal cells using recombinant adenovirus and conjugated adenovirus-DNA coplexes”, Calcif. Tissue Int., 64 (1999), 45-49. This method is particularly advisable for transfection of highly differentiated eukaryote cells with endogenous hormone receptor according to 1. a), since these cells, as opposed to the cell lines without endogenous hormone receptor as defined in 1. b), cannot be sufficiently transfected with conventional transfection techniques. It is of advantage that presently, according to German genetic law, only S1 regulations are compulsory for this method, whereas S2 regulations are compulsory for the “Recombinant Adenovirus” transfection technique.

The principle of the invention is explained using materials with steroid hormone activity as example, without, however, restricting it to these. The hormone active component(s) of the material, termed “ligand” in the following, are added to the transfected cell—(e.g. through addition of the material to the cell culture medium)—and form a ligand-receptor complex with the endogenous and/or recombinant steroid receptor (e.g. glucocorticoid receptor, estrogen receptor, androgen receptor, mineralocorticoid receptor, progesterone receptor, etc.). This ligand-receptor complex binds not only to the HRE sequence of the nd genous DNA in the nucleus, but also to the HRE sequences of the promotor in the reporter expression plasmid resulting in expression of the reporter protein encluded by the respective downstream gene. These reporter proteins do not occur naturally in the eukaryote cells as they are used here. Thus, the formed quantity serves as a direct quantitative measure of hormone activity of the examined material. Examples for such reporter proteins are the enzymes luciferase and β-galactosidase or green-, red-, yellow-fluorescent proteins (GFP, RFP, YFP), which can be quantified easily with existing devices using established protocols and commercially available kits, luminescence reaction in a luminometor or fluorescence reaction in FACS (Fluorescent Activated Cell Sorting). The “Dual Luciferase Reporter Assay System™” (available from Promega, Madison, Wis., USA; cat. no. E1916) is a commonly used protocol for luciferase measurement enabling standardisation of enzyme determination based on the number of successfully transfected cells (via measurement of constitutively expressed renilla luciferase). For the determination of GFP, RFP and YFP in a FACS apparatus (e.g. FACSCalibur, Becton Dickinson, Heidenberg, Germany), standardisation occurs due to the fact that the fluorescence signal can only be transmitted by successfully transfected cells.

Some cells with endogenous hormone receptors according to 1. a) contain different endogenous hormone receptors: HepG2 as well as CC-1, HTC and other “hepatoma tissue cells” (non-hepatoma- or hepatoma lines of human or rat liver), for example, contain glucocorticoid receptor and estrogen receptor; MCF-7 (human breast cancer cell lines) even contain estrogen receptor, glucocorticoid receptor and androgen receptor. However, when discrimination has to be made between the quality of the various steroid hormone activities —e.g. activity of glucocorticoid, estrogen, androgen, mineralocorticoid or gestagen etc.—in the material to be examined, cells without endogenous hormone receptor according to 1. b) are used. These are transfected with the respective hormone receptor expression plasmid, i.e. a construct (e.g. pGR, pER, pAR, pMR, pPR, etc.) coding for the glucocorticoid, estrogen, androgen, mineralocorticoid, progesterone receptor etc. In all cases, a suitable hormone sensitive reporter gene plasmid for all of the listed activity qualities, excl. estrogen active materials, is, for example, GRE (“glue ocorticoid responsive elements”)-Luc with a GRE2-TATA promotor which codes for luciferase. This plasmid (pLCO546A) reacts notably more sensitiviely and thus more strongly to inducing materials with hormone activity than the commonly used plasmid with an MMTV-GRE promotor. For estrogen active materials, ERE-Lu with an ERE-TATA promotor, which also encodes for luciferase, is an example of a suitable plasmid. Suitable cells/cell lines, i.e. without any endogenous hormone receptor, are, for example, CV-1 and COS-1, COS-7 (monkey kidney cells), and especially yeast cell lines which are proven to be most suitable for expression of various genetic products, incl. various “steroid-like receptors” (McEwan IJ: “Investigation of steroid receptor function in the budding yeast Saccharomyces cerevisiae”, FEMS Microbiol. Lett. 179 (1999), 183-4). All these listed as well as other suitable cell lines as defined in 1. a) and 1. b) are commercially available with detailed definition from various cell banks. The following is a selection of suppliers:

ATCC (American Type Culture Collection), 1081 University Boulevard, Manassas (Va.), 20110-2209, USA

DSMZ (Human and Animal Cell Cultures), German Collection of Microorganisms & Cell Cultures, Mascheroder Weg 1b, Braunschweig, D-38124, Germany

ECAAC (European collection of Cell Cultures), CAMR Centre for Applied Microbiology & Research, Porton Down, Salisbury, Wlitschire (UK), SP4 0JG, UK

Since yeast cells, unlike eukaryote cells, posses a cell wall, the above mentioned transfection technique is not suitable. For transfection of these cells, other but well-known procedures are suitable, e.g. the lithium-acetate procedure (Gietz RD et al.: “Studies on the transformation of intact yeast cells by the LiAc/SS-DNA/PEG procedure”, Yeast, 11 (1995), 355-60).

Cotransfection of cells (see 1.a) with a hormone receptor expression plasmid considerably increases the measuring sensitivity of the transfected cell: in addition to the naturally present (“endogenous”) steroid receptors, further “recombinant”, steroid receptors are produced. Consequently, the cells react to even lower concentrations of hormone active materials, thus providing a more sensitive measurement system. Cotransfection is therefore an essential element of this invention.

The bioassay—as per this invention—for the qualification and quantification of materials/molecules and th r material in terms of, for example, suspected steroid hormone activity, steroid hormone agonism and possible antagonism is superior to other assays used for material quantification for another reason. As already mentioned, in the natural and technological environment, there are numerous materials and chemical compounds as well as their, often unknown, metabolites with steroid hormone activity (e.g. phyto-estrogens, pesticides such as DDT and its still ubiquitously present chemical derivatives which act like estrogens). Frequent and important uses of the invention include the investigation of:

1. Known and unknown androgen derivatives, e.g. anabolic agents which are employed by many top athletes as doping agents.

2. Individual estrogen active materials and/or compounds of physiological origin (e.g. human and bovine serum), in foodstuffs, cosmetics and other pharmaceutical products as well as in several technological-synthetic “Permanent Organic Pollutants” (POP).

Suitable for both uses are, for example, COS-1, COS-7, HeLa (undifferentiated, aneuploide cell lines from a rapidly growing human cervical carcinoma), MCF-7, CHO (“Chinese hamster ovary”) cells, and especially yeast cell lines (see above) which are contransfected with an androgen receptor coding or an estrogen receptor coding expression plasmid and a luciferase reporter gene construct which contains GRE2-TATA or ERE-TATA promoter. The principles of use and the methods of assay are as described above.

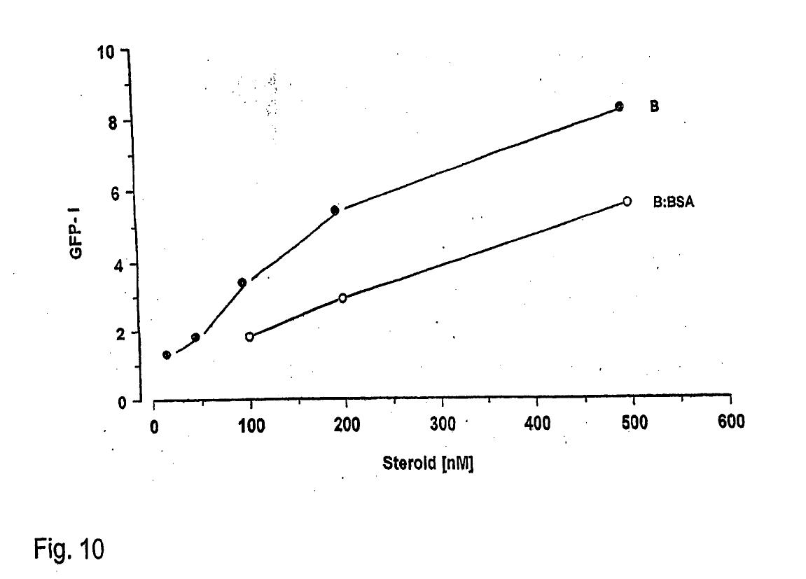

In the following, the invention is explained using a number of specific examples with accompanying illustrations to enable better understanding of the invention. However, its uses are not restricted to these examples. In the following, labelling of the y-axis with “Luc-I” or “GFP-I” (FIG. 10) always means x-time induction (as opposed to control, i.e. without addition of hormone active material to cells) of luciferase or green fluorescent protein illustrated are:

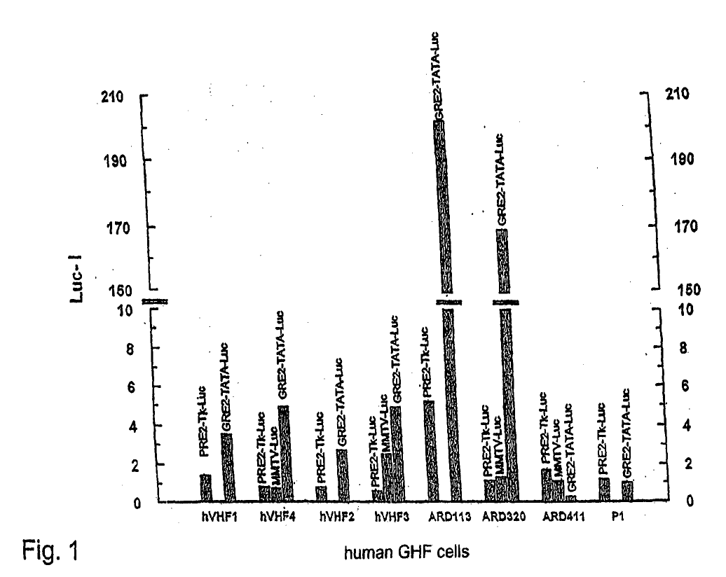

FIG. 1: Androgen induced luciferase activity in human genital skin fibroblasts (GSF) in culture transfected with three different luciferase gen constructs of different androgen sensitivity (“Lu ”): PRE2-Tk-Luc, MMTV-Luc, GRE2-TATA-Luc

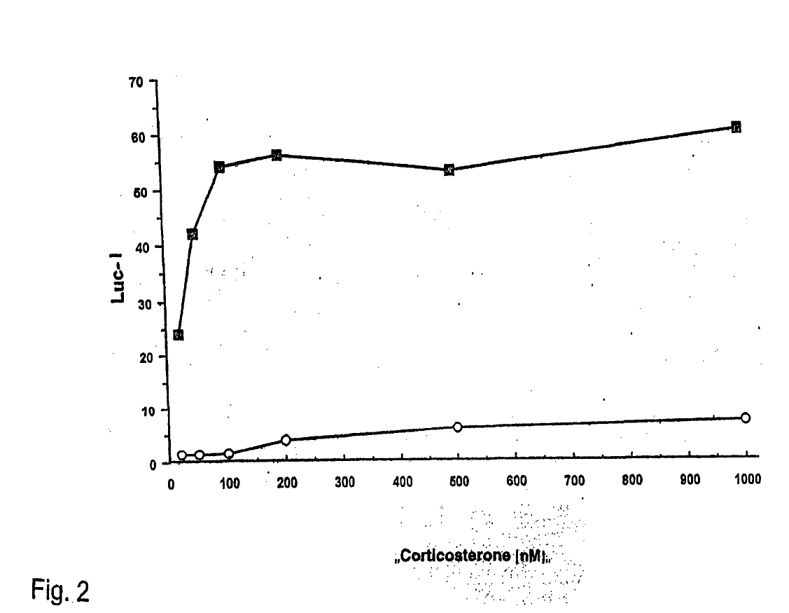

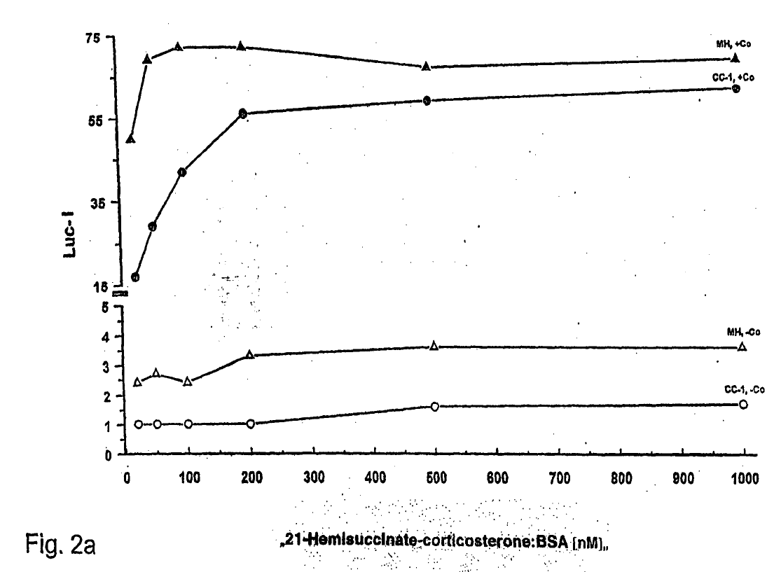

FIG. 2: Dose-dependent effect of a steroid hormone with known glucocorticoid (GC) activity: corticosterone induced luciferase activity in CC1 cells without (o) and with (▪) coexpression of the glucocorticoid receptor.

FIG. 2a: Differing dose-dependent effect of a putatively inactive steroid hormone: 21-hemisuccinat-corticosterone:BSA induced luciferase activity in CC1 and MH 3924 cells without (−Co) and with coexpression (+Co) of the glucocorticoid receptor.

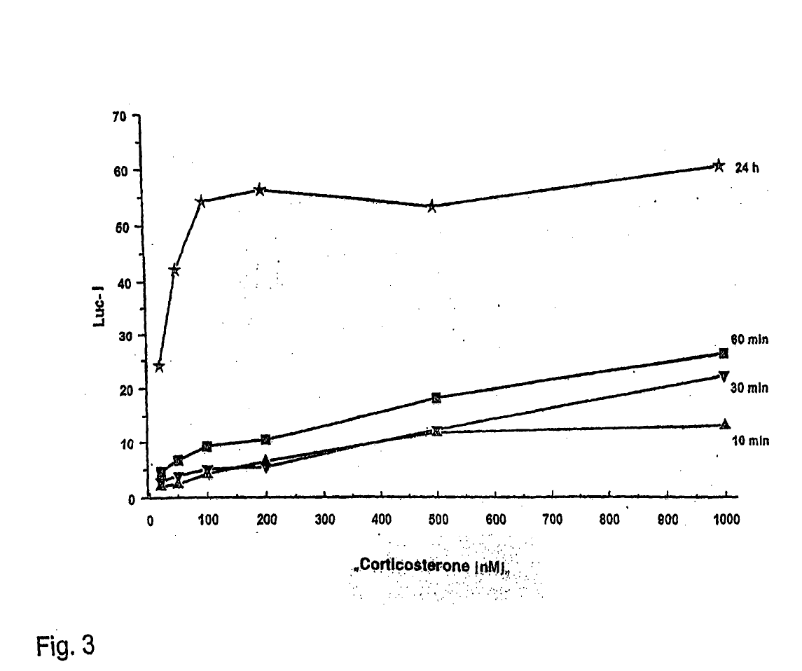

FIG. 3: Induction time dependent dose-dependent effect of a GC active steroid hormone: corticosterone induced luciferase activity for various induction times in CC1 cells.

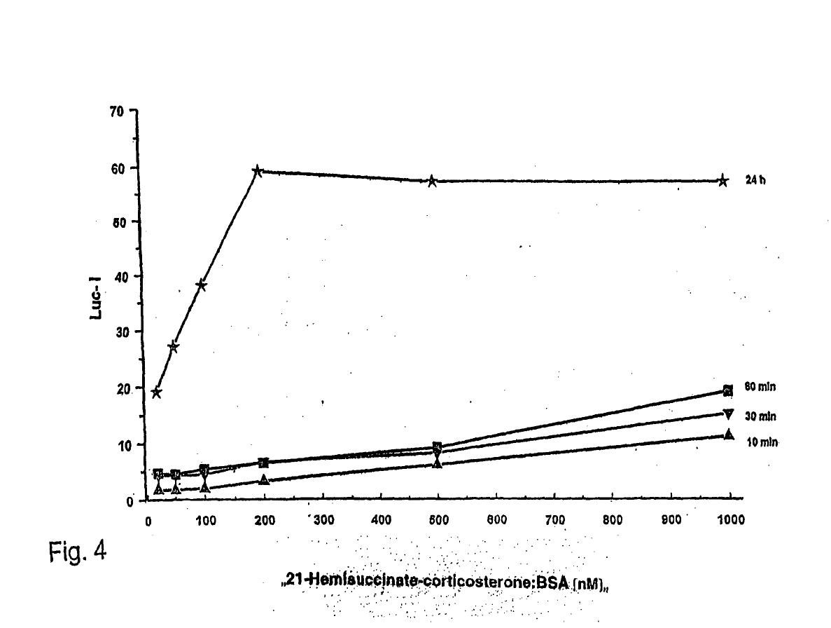

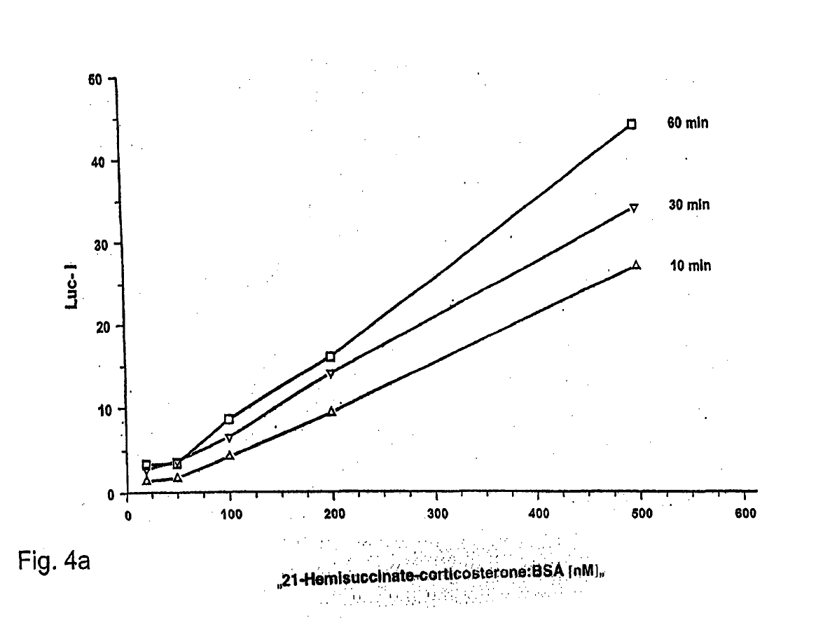

FIG. 4: Induction time dependent dose-dependent effect a putatively inactive steroid hormone: 21-hemisucciant-cortiocosterone:BSA induced luciferase activity in CC1 cells.

FIG. 4a: Short-term induction dependent dose-dependent effect a putatively inactive steroid hormone: 21-hemissucinat-corticosterone:BSA induced luciferase activity in MH 3924 cells.

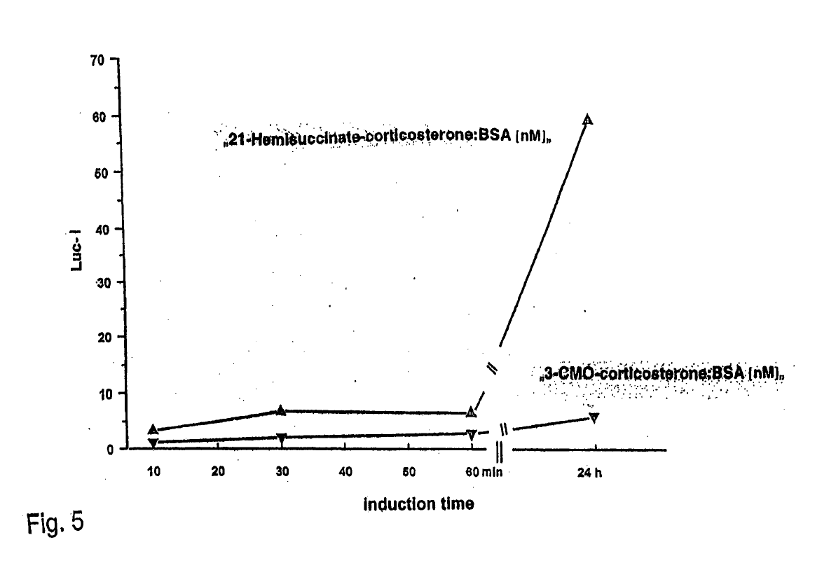

FIG. 5: Induction time dependent effect of steroid derivatives with weak and strong GC activity: induction of luciferase activity in CC1 cells by 3-CMO-corticosterone:BSA or 21-hemisuccinat-corticosterone:BSA.

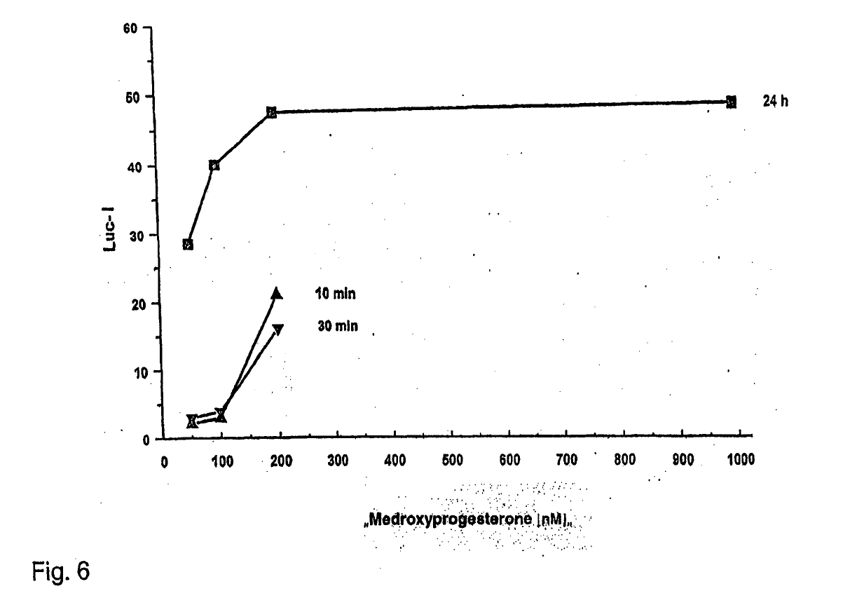

FIG. 6: Induction time dependent dose-dependent effect a steroid hormone with unknown GC activity: induction of luciferase activity in CC1 cells by 6α-Methyl-17α-hydroxy-progesterones (medroxyprogesterone).

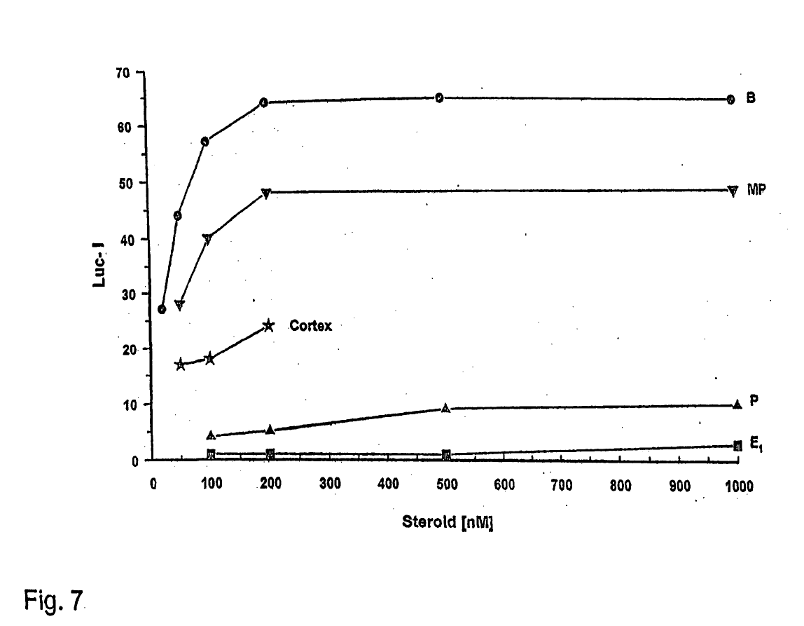

FIG. 7. Dose-dependent effect of various steroid hormones with GC activity of differing strength: induction of luciferase activity by cortiscosterone (B), medroxyprogesterone (MP), estrone (E1), progesterone (P) and 17α-21-dihydroxyprogesterone (Cortex) in CC1 cells.

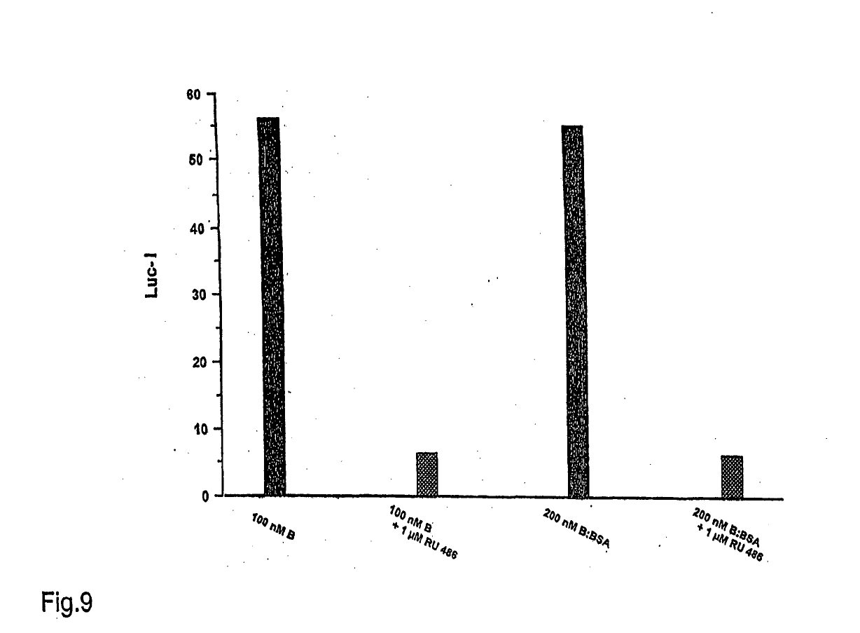

FIG. 9: Dose-dependent effect of various steroid hormones with GC activity of differing strength: induction of luciferase activity by corticosterone (B), medroxyprogesterone (MP), estrone (E1), progesterone (P) nd 17α-21-dihydroxyprogesterone (Cortex) in MH 3924 cells.

FIG. 9: Antagonistic effect of a synthetic steroid hormone on GC activity: inhibition by RU 486 (mifepristone™) of luciferase activity induced in CC1 cells by corticosterone (B):BSA (B:BSA).

FIG. 10: Dose-dependent effect of GC active steroid hormones: corticosterone (B) and 21-hemisuccinat-corticosterone:BSA (B:BSA) induced green fluorescent protein (GFP) activity in MH 3924 cells, stably transfected with a MTV-GFP reporter gene construct and transiently transfected with a glucocorticoid receptor expression plasmid.

The transient transfection method employed in the experiments illustrated in FIGS. 1 through 10 was the “Non-Recombinant Adenovirus-Polylysine” technique.

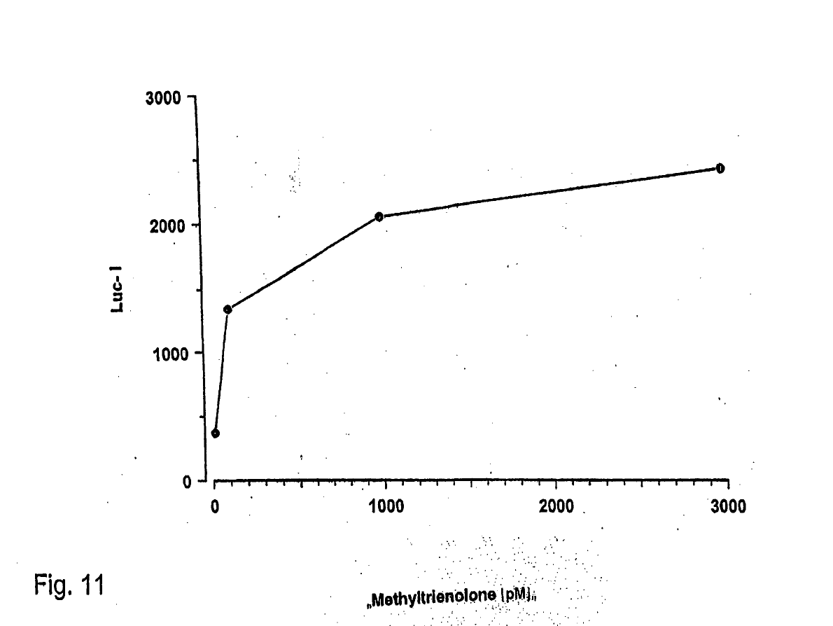

FIG. 11: Dose-dependent effect of a synthetic androgen with anabolic effectiveness: induction of luciferase activity by R1881 (methyltrienolone) in HeLa cells, cotransfected with an androgen receptor expression plasmid. Transfection method in this experiment was the DOTAP method (Boehringer, Mannheim, Germany).

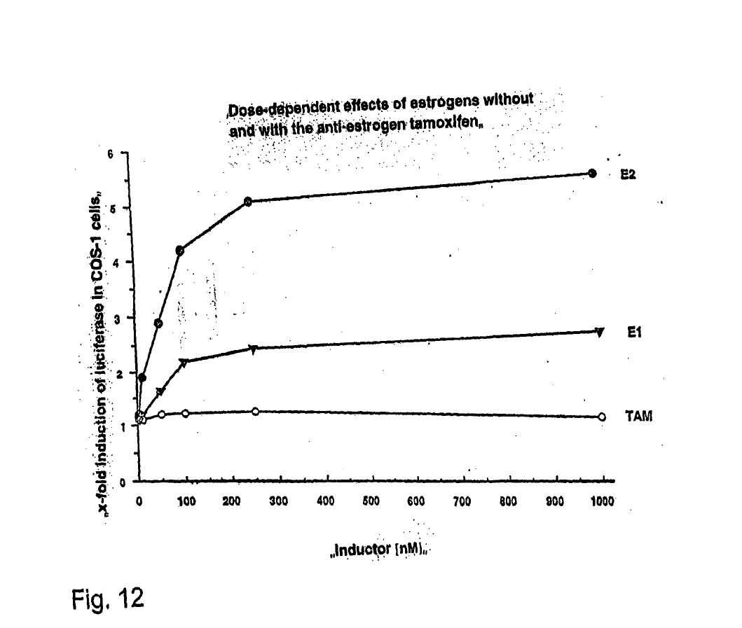

FIG. 12: Estrogen activity determination of estradiol (E2) and estrone (E1) via cotransfected COS-1 cells (example 13). Transient transfection method in COS-1 cells was the Superfact method of Qiagen, USA.

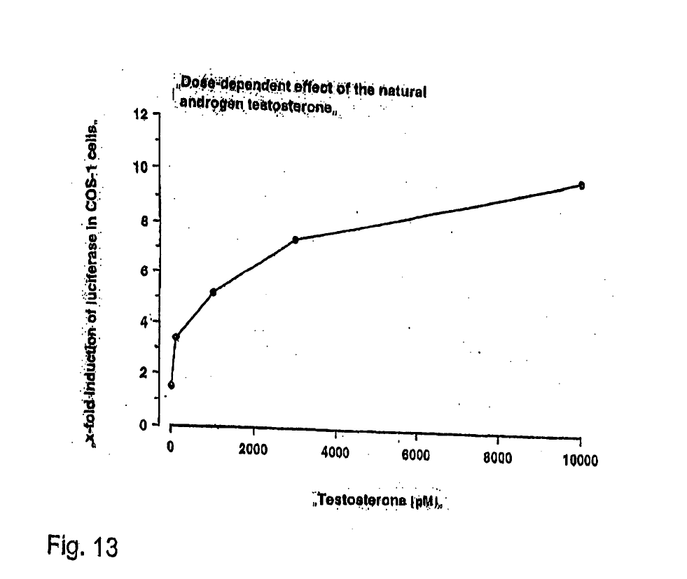

FIG. 13: Androgen activity determination of testosterone with cotransfected COS-1 cells (example 14).

### EXAMPLE 1

### Sensitivity of Assay with Cells, Transfected with Androgen Sensitive Reporter Gene Constructs of Various Strength

As shown in FIG. 1, human genital skin fibroblasts (GSF) in a culture with three different luciferase constructs (“Luc”) were transiently transfected with glucocorticoid responsive element promotors (GRE promotors) of varying androgen sensitivity (with respect to their nucleotide sequence, GRE, glucocorticoid responsive elements, are identical to PRE, “Progesterone Responsive Elements”, and ARE, “Androgen Responsive Elements”). The only steroid hormone receptors present in GSF are androgen receptors (AR). Mibolerone, which is used in this experiment, is a synthetic androgen with anabolic effectiveness. 24 h incubation of the cells with mibolerone (2 nM) results in luciferase activity of varying strength —depending on the origin of GHF (patients with phimosis, “VHF”, or androgen resistance, “ARD-” and “P1”)—inducibility. Obviously, GE2-TATA-Luc is the most sensitive reporter gene construct of the ones used here (for comparison, see also example 10 with a MMTV promotor reporter gene construct as well as FIG. 10).

### EXAMPLE 2

### Assay for the Determination of Glucocorticoid Activity using CC1 and MH 3924 Cells without and with Cotransfection

FIG. 2 displays determination of glucocorticoid activity of corticosterone as well as of a corticosterone derivative bound to a “bovine serum albumin” (BSA) using CC1 cells or MH 3924 cells in culture, both transiently transfected with GRE2-TATA-Luc. These cell lines contain endogenous glucocorticoid receptors. Here, steroid concentration—in this case corticosterone concentration—determines the extent of glucocorticoid activity. Obviously, cotransfection with a recombinant glucocorticoid receptor expression plasmid considerably increases the GC- sensitivity of the cells (see also FIG. 2a). The latter may also be improved by choosing a more sensitive cell line (FIG. 2a). nM (10⁻⁰) stands for nanoMol/litre. Induction time: 24 h (see also example 12).

Thus, the following examples always concern transit ntly cotransfected CC1or MH 3924 cell.

### EXAMPLE 3

### Assay for the Determination of Glucocorticoid Activity using CC1 Cells: Influence of Hormone Concentration and Induction Time

As shown in FIG. 3, the measuring system “cotransfected cell” is so sensitive that a material—in this case corticosterone—will display their inherent hormone activity either at a very low concentration with a long induction time (e.g. 24 h) or at a higher concentration with a very short induction time (e.g. 10 min).

### EXAMPLE 4

### Assay for the Determination of Glucocorticoid Activity of a Corticosterone Derivative using CC1 cells and MH 3924 cells

FIG. 4a and FIG. 2a show how in cotransfected CC1 cells luciferase is induced by a corticosterone derivative which was bound to a bovine serum albumin (BSA). Corticosterone molecules tightly bound within this formulation are definitely responsible for this proven glucocorticoid activity. Therefore, the assay of the invention can determine even slight glucocorticoid activity. This is also shown in FIG. 4a where luciferase induction is easily measurable after a short term induction of 10 to 30 min with a very small concentration of corticosterone:BSA (≦50 nM).

### EXAMPLE 5

### Assay for the Determination of Glucocorticoid activity of Two Different Corticosterone Derivatives using CC1 cells

FIG. 5 shows how a comparison of two different corticosterone:BSA derivatives (21:BSA, 3:BSA, each 200 nM), regarding as being inactive, but proved to possess strong or weak corticosteroid activity, is possible using CC1 cells according to example 4. The difference is particularly distinct with a long induction time. Obviously, even slight differences in the activity of both steroid derivatives can be determined by the assay of this invention within relatively short determination periods—a desirable characteristic for, e.g., “doping” tests or quick tests.

### EXAMPLE 6

### Assay for the Determination of Glucocorticoid Activity of a Synthetic Gestagen using CC1 cells

Here, the synthetic gestagen medroxoxyprogesterone (6α-Methyl-17α-hydroxy-progesterone) was examined using CC1 cells. Surprisingly, a glucocorticoid activity of this material was detected. Previously, no such hormone activity of this material was known. Clinical use of this gestagen includes mainly hormone dependent tumours and endometroisis, but also hormone replacement therapy in post-menopausal women. It is also a component of some depot formulas of oral contraceptives. This —unexpected—hormone activity, which could be determined with the assay of the invention, can be used to predict or determine side effects of such synthetic hormones. Determination by other methods is more extravagant or uncertain. Results are displayed in FIG. 6.

Otherwise, procedures were the same as in example 5—induction times of 10 to 30 min are also sufficient.

### EXAMPLE 7

### Assay for the Determination of Glucocorticoid Activity of Various Steroid Hormones using CC1 Cells

FIG. 7 shows comparison of glucocorticoid activity of various steroid hormones with different concentrations (induction time: 24 h). As expected, corticosterone (B) and medroxyprogesterone (MP)—see example 6—are very active, whereas the natural estrogen esterone (E1) is inactive. However, the natural gestagens progesterone (P) and, most of all, cortexolone (17α-, 21-dihydroxy-progesterone), both classified as glucocorticoid antagonists, display a significant glucocorticoid activity—see also FIG. 7a: “higher sensitivity of MH 3924 cells”.

### EXAMPLE 8

### Assay for the Determination of Glucocorticoid Activity of Various Steroid Hormones Using MH 3924 Cells

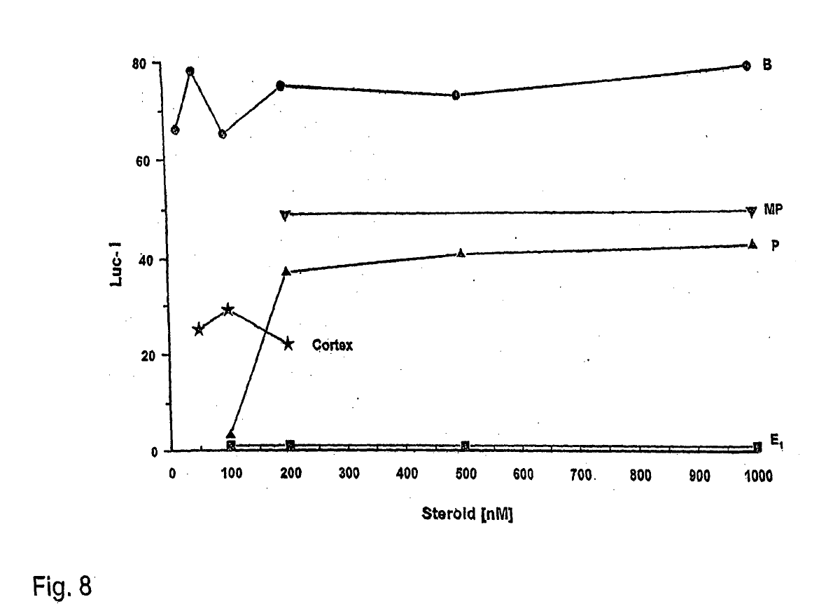

Determination was carried out as in example 7, however, with MH 3924 cells. The response of these cells to glucocorticoid active hormones is distinctly more sensitive than the response of CC1 cells. This is particularly true for progesterone, but also for corticosterone—see FIG. 8 as well as FIG. 7a.

### EXAMPLE 9

### Assay for the Determination of Glucocorticoid Antagonistic Activity of Mifepristone with CC1 Cells

FIG. 9 shows the glucocorticoid antagonism of RU 38486, also known as mifepristone, an essential component of the so-called “abortion pill”. The luciferase inducing effect (glucocorticoid agonism) of corticosterone and 21-hemisuccinat-corticosterone:BSA is extensively blocked by RU 38486—i.e. luciferase induction is inhibited.

### EXAMPLE 10

### Assay for the Determination of Glucocorticoid Activity Using Stably and Transiently Transfected MH 3924 Cells

MH 3924 cells were transiently cotransfected with a glucoorticoid receptor expression plasmid and stably cotransfected with a GFP reporter gene construct containing the MMTV-GRE promotor (induction time:24 h). The result is displayed in FIG. 10. The considerably lower sensitivity of this reporter gene compared to GRE2-TATA-Luc becomes obvious when comparing FIG. 3 with FIG. 4 (see also FIG. 1).

### EXAMPLE 11

### Assay for the Determination of Androgen Activity of a Synthetic Androgen Using HeLa Cells

FIG. 11 show the determination of androgen activity of methyltrienolon (R1881), a synthetic anabolic-androgenic steroid (AAS), via luciferase induction in cultivated HeLa cells. This androgen receptor free cell line was transiently cotransfected with GRE2-TATA-Luc and a recombinant androgen receptor expression plasmid. Hormone activity, i.e. anabolic potency of R1881, can be detected already at a concentration of 10 pM (pM (10⁻¹²)=picoMol/litre. Induction time: 24 h).

### EXAMPLE 12

### Bioassay Using CC1 Cells (Permanent Rat Liver Cell Lines)

Reporter gene construct: GRE2-TATA-Luc (pLCO546A); luciferase (Luc) gene with a minimal glucocorticoid inducing GRE promotor (GRE-TATA)

Constitutive reporter gene: renilla luciferase (pRL-TK vector) with herpes simplex virus thymidinekinase (HSV-TK) promotor (available from Promega, Madison, Wis., USA),

GR expression plasmid: pSTC-rGR: for GR of rat with a constitutive cytomegalus virus (CMV) promotor

GC activity of a material: corticosterone as Luc inducing hormone

Illustration of measurement results: FIG. 2 and FIG. 3

Background of transfection method:

The replication-defect adenovirus type 5 (Adv5, strain DL-312) lacks the “early region” genes E1a and E1b. Reproduction of viruses took place in stably transfected human 293 embryonic kidney cells (293 cells) which complement the missing genes. Following isolation of viruses via a CsCl-density-gradient centrifugation, poly-L-lysine (pLys; Sigma, St. Louis, Mo., USA) was covalently bound to the virus capsule via the 1-ethyl-3-(dimenthyl-aminopropyl)carbodiimid (EDC) technique (Cristiano et al.: Proc. Natl. Acad. Sci. USA. 90: 11548-11552).

Cell culture:

Cell medium used for cultivation of cells is DMEM (Dulbecco's Modification of Eagle's Medium; available as callgro® from M diatech Inc., Park Centre, H rnd n, Va., USA) plus 10% fetal calf serum. For transient transfection, cells are transferred 24 hr prior to transfection from confluently grown culture dishes on 12-well culture plates to 2.5*10⁶ cell per well. During this 24 hr period the cells grow in a medium with steroid hormone-free fetal calf serum. Transfection is carried out when cell have reached a cell density of approximately 60% to 90% confluence. For transfection, the medium is washed and then replaced with phosphate buffer with 0.5 ml medium without fetal calf serum.

The following pipetting scheme is used:

The transfection solution for each well is prepared as follows:

14.2 μl HEPES buffer, pH 7.3

0.5 μl GRE2-TATA-Luc-DNA (100 ng/μl), rGR-DNA (50 ng/μl) and

pRL-TK-DA (5 ng/μl)

2.25 μl adenovirus-pLys solution with 5.7*10¹⁰ particles/ml

These are mixed and incubated in darkness at room temperature for 30 min. Following addition of 7.55 μl pLys-HBS solution in a DNA: Lys weight ratio of 1:1.3; incubaton is continued for another 30 min in darkness.

Transfection and induction:

Cells are incubated in 25 μl of this transfection solution for two hours. The transfection medium is then replaced by 1.5 ml fresh medium, immediately afterwards, or after two to three hours, 12.5 μl steroid hormone solution is added for induction of luciferase production. Blank batches contain hormone fee phosphate buffer with 0.1% ethanol.

For the hormone solution, the enthanol stock solutions are evaporated in a Speed-Vac in glass tubes at 50° C. and 1 atm. Next, they are redisolved in 3 μl ethanol and diluted with phosphate buffer. The given steroid concentrations of 20 nM through 1,000 nM (double values) are the final concentrations in the medium. The ethanol content is not higher than 0.1 %. The blank batch (double value) contains no hormone. Cells are exposed to corticosterone for a maximum period of 24 hr. For short-term inductions of 10, 30 or 60 min, the steroid-containing medium is replaced by a steroid-free one. Cells are incubated for 48 hr with one medium exchange, and after 24 hr with a second hormone addition for 10, 30 or 60 min. Finally, cells are lysed with 200 μl lysi buffer (see below) p r w ll for 20 min at room temperature and shaken occasionally. Lysed cells are stored at −80° C. n culture plates prior to measurement of luciferase activity.

Analysis of reporter signals and determination of luciferase activity

For measurement of luciferse activity, the thawed lysate is transferred to Eppendorf tubes, vortexed for 10 sec and centrifuged at room temperature for 2 min at 14,000×g. The supernatant is vortexed once more and then used in the enzyme test. Luc activity may be measured with any luminometor, e.g. BIOLUMAT LB 95000 T (Berthold, Wildbad, Black Forest, Germany) according to the protocol for the “Dual-Luciferase Reporter Assay System™” (available form Promega, Madison, Wis., USA).

During the enzyme test, activity of firefly luciferase and, immediately afterwards, of renilla luciferase is measured. For this purpose, 10 μl lysate is mixed with 50 μl LAR buffer and light signals are measured in the luminometor. The measuring mode is set at 10 sec, 25° C., Integer. Immediately after that, 50 μl “Stop&Glow” buffer is added, the batch is firmly vortexed for 5 sec and measured again in the luminometor 10 sec following addition of buffer. For determination of the background of the luminometor, both luciferases in the lysate of the respective cells, i.e. transfected either only with firefly luciferase reporter gene or only with renilla luciferase reporter gene, are measured.

The quantity of the induced luciferase for each culture batch is calculated as a multiple of the firefly per renilla luciferase activity of the test batch in correlation with the respective quotient of the hormone free blank batch. For standardization of the different transfection and induction experiments, the x-times induction of each batch is correlated with the batch of 100 nM corticosterone. Mean values of Luc_{(Std.)} are used for evaluation and illustration. Final values result form the following calculation:

$$\begin{matrix}{Luc}_{( 100 )} = \frac{{Luc}_{F + ( 100 )} * {Luc}_{{R}^{-}}}{{Luc}_{R + ( 100 )} * {Luc}_{{F}^{-}}} & ( 1 ) \\ {Luc}_{( N )} = \frac{{Luc}_{F + ( N )} * {Luc}_{{R}^{-}}}{{Luc}_{R + ( N )} * {Luc}_{{F}^{-}}} & ( 2 ) \\ {Luc}_{( Sid )} = \frac{{Luc}_{( N )} * {Luc}_{( Mw ( 100 ) )}}{{Luc}_{( 100 )}} & ( 3 )\end{matrix}$$

Luc₍₁₀₀₎=induction of Luc by 100 nM corticosterone in a single experiment, standardized firefly-Luc/renilla-Luc

Luc_{(N)}=induction of Luc by unequal 100 nM corticosterone in a single experiment, standardized firefly-Luc/renilla-Luc

Luc_{(Std)}=standardized induction of Luc in a single experiment

Luc_{(F)}=Luc values of firefly-Luc with different steroid at various concentrations

Luc_{(R)}=Luc values of firefly-Luc without steroid

Luc₍ₘ₎=Luc values of renilla-Luc with different steroids at various concentrations

Luc₍ₙ₋₎=Luc values of renilla-Luc without steroid

mv=mean value

13. Bioassay using COS-1 cells (permanent monkey kidney cell line)

Transient transfection: SuperFect Transfection Reagent method (substances and instructions available from Qiagen Inc.; Valencia, Calif., USA)

Reporter gene construct: ERE-E1A-Luc; luciferase (Luc)-gene with a minimally estrogen inducible ERE promotor (ERE-TATA)

Constitutive reporter gene: renilla luciferase (pRL-TK Vector) with herpes simplex virus thymidinkinases (HSV-TK) promotor (available from Promega, Madison, Wis., USA)

ER expression plasmid: pSTC-TK-hER; for human estrogen receptor with constitutive thymidinkinas (TK) promotor

Estrogen activity of: 17β- estradiol and estrone as Luc inducing materials

Estrogen antagonist: tamoxifen as copetitive ER antagonist

Illustration of measurement results: see FIG. 12

COS and CV-1 cells do not contain endogenous steroid hormone receptors. Therefore, transient or stable cotransfection of these cells with a recombinant specific hormone receptor expression plasmid is a prerequisite for performing the bioassay.

Information on the transfection procedure: substances and instructions available from Qiagen Inc., Valencia, Calif., USA.

Cell culture:

Cultivation of COS-1 cells is carried out according to example 12

Pipetting scheme, transfection and induction:

Procedures strictly follow the “Protocol for Transient Transfection of Adherent Cells” (substances and instructions available from Qiagen Inc., Valencia, Calif., USA). Apart from the quantities of SuperFect Transfection Reagent and DMEM medium, as specified in the above mentioned protocol, a mixture of 1.5 μg ERE-E1A-Luc DNA, 1.5 μg pSTC-TK-hER-DNA and 5 ng pRL-TK-DNA is added to the COS-1 cells per well of each 12-well culture plate for measurement batches (double values). The stated steroid concentrations of 1 pM through 1,000 pM as well as the tamoxifen concentration (5 μ) are final concentrations i the medium. In terms of blank batch, 24 hr induction time, lysis of cells, etc., procedures as in example 12 are applied.

Analysis of reporter signals and determination of luciferase activity is carried out as in example 12.

### EXAMPLE 14

### Bioassay with COS-1 Cells (Permanent Monkey Kidney Cell Lines)

Transient transfection: SuperFect Transfection Reagent method (substances and instructions available from Qiagen Inc., Valencia, Calif., USA)

Reporter gene construct: GRE2-TATA-Luc (pLCO546A); luciferase (Luc) gene with a minimally glucocorticoid inducing GRE promotor (GRE-TATA)

Constitutitve reporter gene: renilla luciferase (pRL-TK Vector) with herpes simplex virus thymidinkinase (HSV-TK) promotor (available from Promega, Madison, Wis., USA)

AR expression plasmid: pSTC-hAR: for human AR with consitutive cytomegalus virus (CMV) promotor

Androgen activity of testosterone as Luc inducing hormone

Measurement results are shown in FIG. 13.

The entire bioassay is carried out as in example 13. The quantities used per well for DNA of the reporter gene construct, the AR expression plasmid and the constitutive reporter gene pRL-TK are the same as in example 13. Testosterone concentrations of 100 pM though 10 nM (double values) are the final concentrations in the medium.

As the listed examples demonstrate, choice of a highly sensitive reporter gene construct—e.g., GRE2-TATA-Luc-, a very sensitive cell line—e.g., MH 3924—and additional transfection with a hormone receptor expression plasmid—e.g., pGR—can dramatically increase sensitivity of the measuring system “transfected cell”. This is especially distinct in FIG. 2a, cf: “CC-1, -Co” with “MH, +Co”.

It must be pointed out that cell line MH 3924 (available from PD Dr Doris Mayer, working group hormone effects and signal transduction, Department of Tumour Cell Regulation, German Cancer Research Centre, Im Neuenheimer F Id 280, 69120 H idelberg, Germany) is, in terms of its high sensitivity to glucocorticoid aganists, only one example of various other hepatoma cell lines in rats and humans (obtainable from ATCC, DSMZ, ECACC—se above).

The parameters listed as examples are considered to be optimising elements for an application kit of the bioassay of the invention for qualification and quantification of the steroid hormone agonism and possible antagonism of materials/molecules or mixtures of materials. Experts may choose suitable cell lines and reporter gene constructs according to the respective demands (shelf life, sensitivity, specificity etc.) and the invention's theory. The invention is not at all limited to the examples listed above. Naturally, the invention is not limited to exact construction and procedures as detailed in the above mentioned examples. Instead, a wide variety of modifications are possible without deviation from the idea and extend of protection.

1. Method for the determination of peptide hormone activity or steroid hormone activity of a material or material mixture, characterised by:
a) presentation of at least one initial cell with or without endogenous peptide hormone receptors or steroid hormone receptors
b) transfection of cells with a specific recombinant reporter gene construct able to express a measurable product induced by the hormone activity of the material
c) addition of a material to be examined to transfected cell
d) production of an easily determinable signalling reporter gene translation product by the transfected cell via the inducing ability of the material on the hormone reactive promotor preceding the reporter gene
e) measuring of reporter gene translation product of he transfected cell and
f) determination of hormone activity of the material according to measurement results.

2. Method according to claim 1, characterised by production of transfected cell via:
a) presentation of initial cell with endogenous hormone receptor, which transmits hormone activity of the material
b) transfection of initial cell via a vector adding to the cell a recombinant reporter gene product which is reactive for he respective hormone quality

3. Method according to claim 1, characterised by production of a cotransfected cell via:
a) presentation of initial cell with or without endogenous peptide hormone receptor or steroid hormone receptor and
b) transfection of initial cell via a vector adding to the cell a recombinant reporter gene construct which is reactive for the respective hormone quality and an expression plasmid, which encodes for the respective hormone receptor.

4. Method according to any of a previous claim, characterised by selection of the material to be examined from a group consisting of peptide hormones, steroid hormones (e.g., natural and synthetic glucocorticoid-, mineralocorticoid-, androgen- (incl. anabolic-androgenic agents), estrogen- and gestagen-agonists or -antagonists) and other materials with agonistic or antagonistic hormone activity.

5. Method according to any of a previous claim, characterised by the initial cell being a non-plant eukaryote cell or a yeast cell.

6. Method according to claim 1, characterised by the reporter gene being part of an estrogen reactive luciferase reporter gene construct which is produced via:
Transfection of estrogen target cells (e.g. MCF 7, endometrium cells —primary cells and cell lines -) with a luciferase reporter gene construct (estrogen responsive Luc: luciferase gene with a preceding “minimally” estrogen responsive TATA promotor).

7. Method according to any of a previous claim, characterised by selection of the reporter gene construct from a group consisting of, e.g. luciferase, β-galactosidase, β-glucuronidase, green fluorescent proteins, red fluorescent proteins, yellow fluorescent proteins (GFP, RFP, YFP).

8. Method according to any of a previous claim, characterised by employment of a “non-recombinant adenovirus polylysine” technique for transfection.

9. Method according to any of a previous claim, characterised by employment of, e.g., a HeLa primordial cell (undifferentiated, aneuploide cell line from human squamous epithelial uterus cervix carcinoma), CV-1, COS (monkey kidney cells), LnCap (lymph node cells from carcinoma of the prostate), MCF 7.

10. Determination assay for hormone activity of materials, characterised by that it comprises transfected cells according to any of a previous claim.
